(19) United States
(12) Patent Application Publication
(10) Pub. No.: US 2014/0259837 A1
(43) Pub. Date: Sep. 18, 2014

(54) IV SYSTEM TO ASSIST WITH LINE MANAGEMENT

(71) Applicant: CAREFUSION 303, INC., San Diego, CA (US)

(72) Inventors: Scott Belliveau, San Diego, CA (US); Rizaldy Mata, San Diego, CA (US); Joanne Pester-Dewan, San Diego, CA (US)

(73) Assignee: CAREFUSION 303, INC., San Diego, CA (US)

(21) Appl. No.: 13/830,175

(22) Filed: Mar. 14, 2013

(51) Int. Cl. A61M 5/14; G09F 3/04; A61M 5/142

(52) U.S. Cl. CPC A61M 5/1415; A61M 5/142; G09F 3/04
USPC 40/673; 211/49.1

(57) ABSTRACT

Disclosed is a medical fluid infusion system configured for pumping a fluid to a patient. The system includes one or more modular pump devices, such as infusion pumps, each of which is fluidly attached to an IV bag for infusing a liquid from the IV bag to a patient through at least one fluid line. A pole assembly of the medical fluid infusion system is configured to maintain the pumps and IV bags in a vertical alignment that eliminates or minimizes variations in pumping accuracy due to head pressure between the bags and the infusion pumps.

### BACKGROUND

Medical device intravenous (IV) infusion or pumping systems may utilize a plurality of different medical devices that are distinct stand-alone or independent medical devices. For example, some conventional pumping systems may include several distinct stand-alone infusion pumps. Each pump is coupled to a dedicated IV fluid bag as well as a fluid line (such as tubing with an internal lumen) that connects the IV bag to the patient. The infusion pumps can be stacked atop one another in order to conserves space adjacent the patient's hospital bed. As a result, variations in the vertical position of the bags relative to the vertical position of the pumps can cause discrepancies in pumping accuracy due to the effect of head pressure upon the pumped fluid. Moreover, the fluid lines can often become tangled, which makes it difficult for a medical practitioner to match up an IV bag to its corresponding pump.

In view of the foregoing, in multi-pump IV systems there is a need for devices and methods that facilitate proper management of multiple fluid lines between IV bags and the patient.

### SUMMARY

Disclosed is an IV bag pole, comprising: a vertically-oriented backplane post; a bag hanger rod positioned at an upper region of the backplane post, the rod configured to support a first IV bag at a first IV bag vertical height and a second IV bag at a second IV bag vertical height; a first infusion pump seat on the backplane post, the first infusion pump seat configured to support a first infusion pump at a first infusion pump height; a second infusion pump seat on the backplane post, the second infusion pump seat configured to support a second infusion pump at a second infusion pump height; wherein the bag hanger rod is inclined at an angle relative to a vertical such that the vertical difference between the first IV bag height and the first infusion pump height is substantially equal to the vertical difference between the second IV bag height and the second infusion pump height.

The details of one or more variations of the subject matter described herein are set forth in the accompanying drawings and the description below. Other features and advantages of the subject matter described herein will be apparent from the description and drawings, and from the claims.

### DESCRIPTION OF DRAWINGS

FIG. 1 is a diagram illustrating a modular medical device system;

FIG. 2 is a front view of a pump and IV bag assembly of the modular medical device system;

FIG. 3 is a perspective view of the pump and IV bag assembly;

FIGS. 4-6 show close-up views of several infusion pumps of the modular medical device system.

FIGS. 7A, 7B, 8A, and 8B show examples of additional embodiments of line management clips.

FIG. 9 shows an embodiment of a line tag.

FIG. 10 is a logic diagram 300 of an exemplary infusion pump

Like reference symbols in the various drawings indicate like elements.

### DETAILED DESCRIPTION

Disclosed is a medical fluid infusion system configured for pumping a fluid to a patient, such as in a hospital environment. The system includes one or more modular pump devices, such as infusion pumps, each of which is fluidly attached to an IV bag for infusing a liquid from the IV bag to a patient through at least one fluid line. A pole assembly of the medical fluid infusion system is configured to maintain the pumps and IV bags in a vertical alignment that eliminates or minimizes variations in pumping accuracy due to head pressure between the bags and the infusion pumps. The pump and IV bag assembly also maintains the fluid lines in an organized arrangement making it easier for a medical practitioner to figure out which IV bag corresponds to which infusion pump.

FIG. 1 is a diagram illustrating a modular medical device system 110 in a clinical setting. In this view, the modular medical device system 110 is coupled to a plurality of infusion pumps 105, each of which is fluidly coupled to one of many IV bags 110 via a respective fluid line, such as tubing. For example, infusion pump 105a is fluidly coupled to an IV bag 110a via a fluid line 115a to form an IV bag/fluid pump set comprised of the IV bag 110a and the infusion pump 105a. In addition, the each infusion pump 105 is fluidly coupled to a respective flow line 120 inserted into a patient 125 so that the corresponding fluids can be delivered to the patient. The modular medical device system 110 monitors and/or controls how fluids from the respective IV bags 110 are delivered to the patient 125.

A pole assembly 130 is configured to support the infusion pumps 105 and the IV bags 110, as described further below. In the illustrated version, the pole assembly 130 supports eight infusion pumps 105, each of which is attached to one of eight IV bags 110. Thus, there are eight sets of IV bag/infusion pump combinations with each IV bag and infusion pump in a set being fluidly coupled via a respective fluid line. It will be appreciated that varying numbers of infusion pumps can be utilized depending on the particular condition of and/or treatment the patient 125.

FIG. 2 shows a front, plan view of the pole assembly 130 while FIG. 3 shows a front, perspective view of the pole assembly 130. The pole assembly 130 includes a base 205 that supports a vertically-extending backplane 210 configured to mechanically couple to and secure one or more infusion pumps 105 along each of a series of pre-defined mounting seats. The mounting seats are the locations along the pole assembly where an infusion pump 105 attaches to the pole assembly. As described further below, the mounting seats are arranged in a vertical pitch that corresponds to a vertical pitch of the IV bags 110 so as to maintain a constant vertical distance between an IV bag and its respective infusion pump for all IV bag/infusion pump sets. The mounting seats can be arranged along a single axis (e.g., a vertical axis as illustrated, etc.) or they can be arranged along two or more axes. The mounting seats can each have one or more mechanical elements to detachably affix the infusion pumps 105 to the backplane 210.

With reference still to FIGS. 2 and 3, an upper region of the pole assembly 130 includes a bag hanger 215 configured to support the IV bags 110 in a predetermined vertical position relative to the infusion pumps 105. In the illustrated version, the IV bags 110 are supported in an inclined or diagonal arrangement with each IV bag positioned next to and vertically lower than (or vertically higher than) an adjacent IV bag. In this regard, the bag hanger 215 includes one or more inclined hanger rods 220 from which the IV bags hang using any of a variety of hanging structures, such as hooks.

As mentioned, each of the IV bags 110 has a corresponding flow line 115 that connects to a respective infusion pump 105. Likewise, each infusion pump 105 connects to a corresponding flow line 120 that connects to the patient 125. It is possible that variations in the vertical positions of the IV bags relative to the vertical positions of the infusion pumps 105 may incur variations in head pressure of fluid between the pumps that may affect the pump accuracy of the infusion pumps. It is therefore desirable for the head pressure to remain relatively constant between all of the IV bag/infusion pump sets in the system. In other words, the vertical distance between an IV bag and an attached infusion pump should be constant for all of the IV bag/infusion pump sets in the system. The disclosed pole assembly maintains a constant vertical distance between all of the infusion pumps and IV bags.

This is described in more detail with respect to two exemplary infusion pumps in FIGS. 2 and 3, specifically infusion pump 105a and infusion pump 105b. Infusion pump 105a is attached to IV bag 110a via infusion line 115a, while infusion pump 105b is attached to IV bag 110b via infusion line 115b. Likewise, fluid line 120a attaches infusion pump 105a to the patient and fluid line 120b attaches infusion pump 120b to the patient. The fluid line 115a enters infusion pump 110a at vertical position A1, while the fluid line 115b enters infusion pump 110b at vertical position B1. A vertical distance of ΔD1 separates the vertical locations A1 and B1. Similarly, the fluid line 115a interfaces with IV bag 110a at vertical position A2, while the fluid line 115b interfaces with IV bag 110b at vertical position B2. A vertical distance of ΔD2 separates the vertical locations A2 and B2. It is desirable that the distances ΔD1 and ΔD2 be equal or substantially equal so as not to introduce variations in head pressure between different infusion pumps of the system. This would be the case for all the IV bag/infusion pump sets in the system.

Another way of characterizing this is that the vertical distance between A1 and A2 (which determines the head pressure for infusion pump 105a) is equal to the vertical distance between B1 and B2 (which determines the head pressure for infusion pump 105b). This is the case for all IV bag/infusion pump sets in the system. The incline relative to vertical (or horizontal) of the hanger rods 220, as well as the positions of the IV bags 110 along the hanger rods 220, is selected to achieve such a state.

Toward this end, the hanger rods 220 are inclined in a manner that maintains a pitch between adjacent IV bags that is equal to or substantially equal to the pitch between adjacent infusion pumps. In other words, the incline of the hanger rods 220 is such that the vertical distance between adjacent bags is equal to the vertical distance between adjacent pumps for all the IV bag/infusion pump combinations in system. The pitch between the bags is therefore equal to the pitch between the infusion pumps. Thus, the delta in vertical distance between adjacent pumps is equal to the delta in vertical distance between adjacent bags. This maintains an absolute vertical height difference between a given pump and corresponding IV bag for all the IV pump and bag combinations on the system.

It should be appreciated that the incline of the hanger rods 220 can vary to suit various spatial and ergonomic requirements. An inclined orientation is advantageous in that it can reduce the absolute vertical height of the system, although the incline can be close to a completely vertical incline.

### Management of Flow Lines

Given the quantity of infusion pumps and corresponding flow lines, it is possible that the flow lines 115 or 120 can become tangled or unruly particularly as the quantity of medical infusion pumps 105 increases. There is therefore a need for a mechanism for managing the flow lines 175 so as to improve accessibility and visual appearance of the multiple flow lines 115 and 120.

FIGS. 4 and 5 show enlarged views of some of the infusion pumps 105 of the system showing some of the fluid lines 115 and 120 that are included in the system. At least one fluid line management clip 405 is coupled to the pole assembly for arranging the fluid lines in a managed position and reduce the likelihood of the fluid lines becoming tangled. The management clips 405 desirably maintain the fluid lines in a predetermined spatial arrangement. For example, the management clips 405 may maintain the fluid lines such that, when left unattended, the fluid lines fall generally parallel to one another as shown in FIGS. 4 and 5. Any quantity of management clips 405 can be used. It can be advantageous to have at least one management clip 405 for the lines 115 and one management clips for the lines 120 although it is possible that only the lines 115 or the lines 120 can be tied to a clip.

With reference to FIGS. 4 and 5, each management clip 405 is attached to the pole assembly such as via an arm 410. The arm 410 is sized and shaped such that the management clip 405 is positioned at an ergonomic location relative to the pumps 105. That is, the clips 405 can be positioned to provide a small distance between where the management clip 405 is located and the position of the infusion pumps 105. Each management clip 405 has one or more slots that provide a space where a respective fluid line can be positioned and secured.

FIG. 6 shows a close-up view of a line management clip 405. As mentioned, each management clip 405 includes a line seat or line slot 420 that is sized and shaped to receive at least one fluid line 115. In one version, the slot 420 is sized and shaped to receive only a single fluid line 115 and in another version, the slot 420 is sized and shaped to receive multiple fluid lines. The slots 410 can be shaped so that the fluid line 115 fits snug in the slot such that, once inserted, the fluid line 115 is secured within the slot. For example, the slot 420 may be sized and shaped relative to the size and shape of the fluid line 115 such that the line management clip 405 must be elastically deformed in order to insert the line 115 into the slot. A corresponding elastic deformation to the management clip 405 would have to be performed to remove the fluid line 115 from the slot 420.

Or the slot 420 can be sized and shaped such that it slightly grasps the fluid line 115 once the fluid line 115 is positioned in the slot. This would secure the fluid line 115 in the slot 420 once positioned therein. Although FIG. 6 shows the management clip 405 with four slots, it should be appreciated that the quantity of slots per clip can vary. In addition, one or more line management clips 405 may be attached directly to an infusion pump 105 rather than to an arm 410.

The line management clips 405 can be made of any of a variety of materials.

FIGS. 7A, 7B, 8A, and 8B show examples of additional embodiments of line management clips that can be used in place or in addition to the clips 405. FIG. 7A shows a side view of a first embodiment of a clip 705 that is sized and shaped to retain a single line 115 (or 120) or loop a longer single line multiple times into the channel 710 to prevent the line from touching the floor. FIG. 7B shows a perspective view of the clip 705. The clip 705 has a flat or contoured region 707 that is configured to be affixed to any surface such as surface of one of the infusion pumps 105. A front, bulbous region 710 includes a pair of lips 715 that defines an opening into a channel 710 that is sized and shaped to receive and retain at least one line 115. The front lip 715a is at the tip of an upwardly-extending hook-like structure that covers the channel 710.

The lips 715 can be separated from one another to expose the channel 710 for receipt of a line 115. The lip can have a resilient characteristic such that, after being separated to allow entry of the line 115, the lips spring back into position to retain the line within the channel 710.

The line 115 can be inserted into the channel 710 by an operator. In this regard, the operator may grasp the line 115 and press the line 115 against the front lip 715a until the lip “gives” or otherwise deforms to expose the channel. The line 115 can snap into place within the channel 710 as a result of the pressure exerted onto the lip 715a by the line 115. The line 115 may snap past the lips into the channel 710 with an audible feedback. The line 115 can also be grasped by the operator and be pulled upward and out of the channel 710 with minimal effort.

In another embodiment, shown in FIGS. 8A and 8B, a clip 805 is similar to the embodiment of FIGS. 7A and 7B. In this embodiment, the front lip 815a and back lip 815 are more pronounced in shape than the more widened lips 715 of the previous embodiment. The clip 805 operates in the same manner as the clip 705.

FIG. 9 shows an embodiment of a line tag 905. Each line tag is a substantially planar or sheet-like structure having a pair of openings through which a line 115 can be threaded. The material of the line tag 905 may be flexible or rigid. A perforation or slit 911 is aligned with each opening to provide access to the opening such that a line 115 (or 120) can be inserted into the opening by pushing the line through the slit 911. In an embodiment, there is an audible snap or other sound when a line is pushed into or pulled out of each opening of the line tag 905. The audible snap is caused by the material of the line tag 905 and/or its shape.

A line 120 can be quickly retained within a respective line tag 905 by pushing a portion of the line 115 through the slits 911 so as to separate the slit such that the line 115 threads through the openings as shown in FIG. 9. The line tag 905 is made of a material of sufficient stiffness and rigidity and has a shape such that the line 115 forms a tension against the line tag 905 once the line 115 is inserted into the line tag 905. This tension serves to retain the line tag 905 in a relatively fixed position along the line(s) 115 until the operator exerts sufficient force on the line tag 905 to either release the line 115 from the tag (via the slots 911) or to slide the line tag 905 along the length of the line 115.

One or more graphic lines 913 or other indicators can be printed or otherwise located on the tag 913 to visually delineate separate line tags from one another. In this regard, the line tag 905 can be a single line tag that holds multiple lines 115 (four lines in the example shown), or the line tag 905 can be broken apart (such as via perforations) to separate the line tag 905 into multiple line tags, each of which may retain one or more lines 115. The line tag 905 can have multiple perforations that allow the operator to split off all lines individually, or just split off one line to have a group of 2 or more lines together. FIG. 9 shows the line tag 905 configured to retain four lines 115 although the quantity of lines can vary.

Once one or more lines 115 are retained in a line tag 905 as shown in FIG. 9, an operator can position the line tag 905 at any desired location along the length of the line(s) 115 by simply grasping the line tag 905 and sliding the line tag 905 along the length of the line(s) 115 to the desired location along the line 115.

A plurality of line tags 905 can be assembled adjacent to one another as shown in FIG. 9 so that a plurality of lines can be attached to respective line tags 905. Once inserted into the line tag 905, the line 115 is retained to the line tag 905 until the line 115 is manually separated from the line tag 905. The line tag 905 may be made of a material that can be written upon such as with a pen, pencil, or other writing tool. This permits an operator to label the line tag as needed.

### Exemplary Configuration of Medical Device System

In addition to allowing the medical device modules to be affixed to the system, the backplane 210 can provide non-contact inductive power to one or more of the infusion pumps 105. The backplane 210 can, for each mounting location, comprise an inductor for non-contact powering of an infusion pump 105. A corresponding inductor on the infusion pump 105 can, when the infusion pump 105 is affixed to the mounting seat, be inductively coupled with inductor of backplane 210. In general, energy is sent via an inductive (magnetic) coupling between the inductor of the backplane and inductor of the infusion pump. As a result, there is a wireless (no galvanic contact) energy transfer between inductive backplane 210 and infusion pump 105. Moreover, an electrical galvanic connector, as is typical for powering conventional medical devices, is not required to provide power to infusion pump 105. Use of non-contacting energy transfer avoids metallic contacts between infusion pump 105 and a power source which may be damaged, require special cleaning and pose risk of electrical heating, smoke or fire. Each inductor can be coupled to an induction bus which in turn is connected to a power source (e.g., a wired connection to an outlet, a battery, etc.) to enable the inductive coupling of each inductor.

The backplane 210 can also provide an optical communications interface to one or more infusion pumps 105 via respective optical communications ports and optical transceivers corresponding to each mounting seat. The infusion pumps 105 can have corresponding optical communications ports and optical transceiver, which can be optically aligned with the optical communication port on the backplane when the infusion pump 105 is affixed to the backplane 210 so that a bi-directional data feed can be established between the optical transceivers. Such data can relate to a variety of aspects including, but not limited to, data characterizing operation of the infusion pump 105, data for controlling or otherwise modifying an operating parameter of the infusion pump 105, and the like.

The data transmitted to the backplane 210 can be consumed locally by the system 110 and/or it can be transmitted to one or more remote systems/devices coupled to the system 110 via a wired or wireless communications link. The optical data transceivers can be infrared (IR) data transceivers such that optical data is propagated by IR light as the transmission medium. The optical data transceivers can be coupled to a communications bus that in turn is coupled to a communications interface. The communications interface can, in turn, be coupled to the controller. In addition or the alternative, the communications interface can provide an outward interface for the modular medical device system 110 that provides a wired or wireless connection to other devices and/or networks.

Infusion pump 105 can be any medical device that is compatible for scalability in a modular medical device system. For instance, the modular medical device system 110 can utilize one or more infusion pumps 105 depending on the functionality that is needed for proper care of a patient. Moreover, a modular medical device system 110 can be scaled up to incorporate additional infusion pumps 105 and also scaled down by removing infusion pumps 105. For example, if patient care requires only one infusion pump, then the modular medical device system 110 can include a single affixed infusion pump. Moreover, if patient care requires two infusion pumps, then the modular medical device system 110 can be scaled up to include an affixed additional infusion pump.

Infusion pumps 105 can include, but are not limited to, an infusion pump (e.g., a large volume pump (LVP), a syringe pump), a patient-controlled analgesia (PCA) system, a vital signs monitor (VSM), a bedside blood analyte analyzer (e.g. blood glucose), an Auto-ID module (barcode scanner and RFID), and other devices which measure physiological parameters associated with a patient and/or assist with the clinical care of the patient (and such infusion pumps 105 may not necessarily measure physiological parameters of the patient).

The modular medical device system 110 can also comprise a display unit 133 that provides a unified interface that characterizes the operation of various infusion pumps 105 coupled to the backplane 210. The display unit 133 can comprise a touch screen interface that allows a user to selectively view and alter performance parameters/metrics of individual infusion pumps 105, concurrently view performance parameters/metrics of multiple infusion pumps 105, and additionally orchestrate complex sequences of infusions/operations from multiple infusion pumps 105. The display unit 133 can be affixed to an outer housing of the modular medical device system 130/inductive backplane 210 by a tilt and swivel arm mount that allows the display unit to be moved on different sides of the system 110 and/or to change varying positions (to accommodate different positions/heights of caregivers). The following U.S. patent application describes an exemplary pump system and is incorporated by reference herein in its entirety: U.S. patent application Ser. No. ______ entitled “Modular Medical Device System” (attorney docket no. 45004-038F01US), filed concurrently herewith.

FIG. 10 is a logic diagram 300 of an exemplary infusion pump 105. The infusion pump 105 can include a secondary power source 156 such as a battery or a wired connection to an external power source. For example, the secondary power source 156 can power medical device module 120 when inductor 152 is unable to power infusion pump 105. In one scenario, infusion pump 105 is an infusion pump that is associated with (and fluidly communicating with) a patient. If the patient is moved to another location (e.g., to an x-ray room which is away from inductive backplane 110), then infusion pump 105 must also move with the patient away from inductive backplane 210 in order to continue the current infusion without interruption. Upon a certain distance from inductive backplane 210, inductor 152 cannot be energized by inductive backplane 210 and therefore cannot power infusion pump 105. Accordingly, power source 156 (which may also be charged through inductor 152) is able to provide the requisite power for infusion pump 105. In one variation, the power source 156 is a battery that can keep infusion pump 105 operational in a range of about two to four hours.

Each infusion pump 105 can also include memory 157 and at least one data processor 158. The memory 157 can store instructions for execution by the at least one data processor 158 for use, for example, in the operation of the medical device module in a clinical setting. The memory 157 can also store data relating to the operation of the medical device module such as data characterizing how the infusion pump 105 is used and parameters relating to same (e.g., number of hours operated, thresholds for alerts, etc.). It is noted that when a infusion pump 105 is reattached to the prior inductive backplane or a different inductive backplane, information required to continue the infusion stored in memory 157, without interruption, can be transmitted from the infusion pump 105 to the backplane (and to the controller 140).

Each infusion pump 105 can also comprise an additional communications interface 158 other than the optical data transceiver 154 (in some variations the optical data transceiver 154 may not form part of the infusion pump 105 and so the communications interface 158 may be the only gateway for communication outside of the infusion pump 105). This communications interface 158 can be fixed and/or wireless and be used to communicate to computer networks and peer-to-peer pairing with other devices when the infusion pump 105 is not coupled to the backplane 210. In some implementations, the communications interface 158 can be used in addition or instead of the optical data transceiver 154 when the infusion pump 105 is coupled to the backplane 210. For example, the infusion pump 105 can be seated on the backplane 210 but not have an optical data transceiver. In such a scenario, the communications interface 158 can wirelessly communicate with the controller 140 of the modular medical device system 110 so that the operation of the infusion pump 105 can be monitored and/or controlled by the modular medical device system 110.

The system 110 comprises a controller (which in turn can comprise at least one data processor and memory for storing instructions for execution by the at least one data processor and/or data characterizing or otherwise relating to the operation of medication device modules). The controller can act to individually monitor and/or control the operation of the infusion pumps 105 affixed to the backplane 210 such that the functionality of the infusion pumps 105, alone and/or in combination are increased. In some cases, the controller can orchestrate the operation of multiple infusion pumps 105. For example, certain sequences of operation and/or concurrent operation can be defined amongst the infusion pumps 105. Such an arrangement can permit, for example, coordinated infusion from different fluid sources. Some infusion pumps 105 can have the ability to function fully independent of the controller 140 for the purpose of basic operations. However, the modules acquire more complex abilities and functionality when operating under the command and coordination of the controller.

One or more aspects or features of the subject matter described herein may be realized in digital electronic circuitry, integrated circuitry, specially designed ASICs (application specific integrated circuits), computer hardware, firmware, software, and/or combinations thereof. These various implementations may include implementation in one or more computer programs that are executable and/or interpretable on a programmable system including at least one programmable processor, which may be special or general purpose, coupled to receive data and instructions from, and to transmit data and instructions to, a storage system, at least one input device (e.g., mouse, touch screen, etc.), and at least one output device.

These computer programs, which can also be referred to programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid state memory or a magnetic hard drive or any equivalent storage medium. The machine-readable medium can alternatively or additionally store such machine instructions in a transient manner, such as for example as would a processor cache or other random access memory associated with one or more physical processor cores.

These computer programs, which can also be referred to programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid state memory or a magnetic hard drive or any equivalent storage medium. The machine-readable medium can alternatively or additionally store such machine instructions in a transient manner, such as for example as would a processor cache or other random access memory associated with one or more physical processor cores.

With certain aspects, to provide for interaction with a user, the subject matter described herein can be implemented on a computer having a display device, such as for example a cathode ray tube (CRT) or a liquid crystal display (LCD) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including, but not limited to, acoustic, speech, or tactile input. Other possible input devices include, but are not limited to, touch screens or other touch-sensitive devices such as single or multi-point resistive or capacitive trackpads, voice recognition hardware and software, optical scanners, optical pointers, digital image capture devices and associated interpretation software, and the like.

The subject matter described herein may be implemented in a computing system that includes a back-end component (e.g., as a data server), or that includes a middleware component (e.g., an application server), or that includes a front-end component (e.g., a client computer having a graphical user interface or a Web browser through which a user may interact with an implementation of the subject matter described herein), or any combination of such back-end, middleware, or front-end components. The components of the system may be interconnected by any form or medium of digital data communication (e.g., a communication network). Examples of communication networks include a local area network (“LAN”), a wide area network (“WAN”), and the Internet.

The computing system may include clients and servers. A client and server are generally remote from each other and typically interact through a communication network. The relationship of client and server arises by virtue of computer programs running on the respective computers and having a client-server relationship to each other.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flow(s) depicted in the accompanying figures and/or described herein do not necessarily require the particular order shown, or sequential order, to achieve desirable results. Other implementations may be within the scope of the following claims.

What is claimed is:

1. An IV bag pole, comprising:
a vertically-oriented backplane post;
a bag hanger rod positioned at an upper region of the backplane post, the rod configured to support a first IV bag at a first IV bag vertical height and a second IV bag at a second IV bag vertical height;
a first infusion pump seat on the backplane post, the first infusion pump seat configured to support a first infusion pump at a first infusion pump height;
a second infusion pump seat on the backplane post, the second infusion pump seat configured to support a second infusion pump at a second infusion pump height;
wherein the bag hanger rod is inclined at an angle relative to a vertical such that the vertical difference between the first IV bag height and the first infusion pump height is substantially equal to the vertical difference between the second IV bag height and the second infusion pump height.

2. An IV bag pole as in claim 1, wherein the rod is configured to further support at least a third IV bag at a third IV bag height, and further comprising:
at least a third infusion pump seat on the backplane post, the third infusion pump seat configured to support a third infusion pump at a third infusion pump height;
wherein the rod is inclined at an angle relative to a vertical such that the vertical difference between the third IV bag height and the third infusion pump height is substantially equal to the vertical difference between the second IV bag height and the second infusion pump height.

3. An IV bag pole as in claim 1, further comprising a fluid line clip configured to secure at least one fluid line in a predetermined position.

4. An IV bag pole as in claim 3, wherein the fluid line clip secures multiple fluid lines in predetermined positions.

5. An IV bag pole as in claim 4, wherein the fluid line clip secures the multiple fluid lines such that the multiple fluid lines hang in a parallel orientation relative to one another.

6. An IV bag pole as in claim 1, wherein the backplane post comprises an inductive backplane configured to secure and inductively power a plurality of detachable infusion pumps.

7. An IV bag pole as in claim 6, wherein the backplane post comprises:
a communications interface; and
a control unit to control, via the communications interface, at least one operational parameter of each infusion pump when the infusion pump is secured to the inductive backplane.

8. An IV bag pole as in claim 6, wherein:
the communications interface is coupled to at least one optical data transceiver and a plurality of first optical data transmission ports along the inductive backplane, and
each infusion pump comprises a second optical data transmission port positioned along an optical path with a corresponding first optical data transmission port when the medical device module is secured to the inductive backplane.

9. An IV bag pole as in claim 1, wherein each seat is configured to mechanically secure one infusion pump.

10. An IV bag pole as in claim 1, wherein the seats are arranged along a vertical axis of the backplane.

11. An IV bag pole as in claim 1, further comprising a touch screen display extending outward from the backplane.

12. An IV line tag, comprising:
a planar structure having a first opening and a second opening, the first and second openings configured to receive a fluid line therethrough;
at least one slit in the planar structure associated with each of the first and second openings, the at least one slit capable of being separated for insertion of a fluid line into the opening.

13. An IV line tag as in claim 12, wherein the planar structure can be slid upon the length of a line that is threaded through the first and second openings.

14. An IV line tag as in claim 12, wherein the planar structure is made of a material that can be written upon.

15. An IV line tag as in claim 12, further comprising third and fourth openings configured to receive a second fluid line.

16. An IV line tag as in claim 15, further comprising a perforation on the planar structure that separates the first and second openings from the third and fourth openings.
